(19) United States
(12) Patent Application Publication
(10) Pub. No.: US 2013/0178674 A1
(43) Pub. Date: Jul. 11, 2013

(54) REACTOR AND PROCESS FOR DEHYDRATION OF ETHANOL TO ETHYLENE

(71) Applicants: Hassan Taheri, Hinsdale, IL (US); Yogendra Sarin, Plainsboro, NJ (US); Brian Ozero, Westhampton, NY (US)

(72) Inventors: Hassan Taheri, Hinsdale, IL (US); Yogendra Sarin, Plainsboro, NJ (US); Brian Ozero, Westhampton, NY (US)

(21) Appl. No.: 13/346,407

(22) Filed: Jan. 9, 2012

(51) Int. Cl. C07C 1/24; B01J 7/00

(52) U.S. Cl. 585/300; 585/639; 422/600; 422/630

(57) ABSTRACT

A reactor design and configuration and a process for the catalytic dehydration of ethanol to ethylene where the reactor train is comprised of a multi-stage single reactor vessel or multiple reactor vessels wherein each stage and/or vessel has different length, internal diameter, and volume than the other stages and/or vessels and in addition the stages and/or reactor vessels are connected in series or in parallel arrangement, preferably used with an improved means of introducing the ethanol feedstock and a heat carrying inert gas to the improved reactor train.

### BACKGROUND OF THE INVENTION

1. Field of the Invention

This invention relates to an improved technology of reactor design and configuration and a process for the catalytic dehydration of ethanol to ethylene wherein the reactor train is comprised of a multi-stage single reactor vessel or multiple reactor vessels wherein each stage and/or vessel has different length, internal diameter, and volume than the other stages and/or vessels and in addition the stages and/or reactor vessels are connected in series or in parallel arrangement. Furthermore, this invention discloses an improved means of introducing the ethanol feedstock and a heat carrying inert gas to the improved reactor train.

2. Related Information

Ethylene is the backbone of the petrochemical process industries providing raw materials for many applications including industrial chemicals, consumer products, polymers, plastics, surfactants, etc. This petrochemical feedstock is primarily produced from petroleum resources by the steam cracking of petroleum-derived feedstocks such as heavy naphtha, ethane/propane, or gas condensates. The economics of these processes are greatly influenced by the supply, availability, and price of crude oil and natural gas. In addition, the cracking processes produce a large number of valuable by-products such as propylene, butylene, etc. which require considerable energy per ton of products due to higher cracking temperatures, much more complex processing, and high capital investment to separate, purify, and market all the products so that the process can be economically justified. If a user of ethylene were only interested in producing ethylene, the cracking route is not an advantageous option. Furthermore, the conventional steam cracking produces large quantities of CO₂ (carbon) which is a greenhouse gas.

The catalytic dehydration of ethanol to ethylene is a well-known commercial process for the selective conversion of the ethanol to value-added ethylene. In the 30's and 40's, several ethanol dehydration units were built which remained in operation until the 60's. The world oil crisis of early 70's accelerated the development of ethanol dehydration technologies with several new plants built in the 80's. However, the dehydration process fell out of favor in the mid-90's due to abundant supply and low price of crude oil and natural gas.

Recently, as the biofuels have attracted more attention globally, as prices of crude oil have increased, and as its supply sources have become more unstable and problematic, the alternative route of ethanol dehydration process has again become an important alternative source of ethylene supply. In addition, with the threat to the environment and limited resources in some parts of the world, the ethanol dehydration process is being increasingly competitive with the traditional steam cracking process. Additionally, the sources of raw materials for ethanol supply has increased many folds over the last decade from renewable/sustainable sources such as may be readily obtained by fermentation from such diversified bio-resources as sugar cane, corn, agricultural and cellulosic biomass, or algae based feedstocks.

The ethanol dehydration reaction basically is characterized by the removal of a water molecule from ethanol and as such is highly endothermic. A significant amount of heat (energy) is thus required to initiate and sustain the reactions to completion. The economic production of ethylene by this process largely depends on the efficient conversion of ethanol feedstock and high selectivity and yield of the ethylene product. In addition, it is critical to limit the formation of by-products which will complicate the purification of the product and its downstream application into high value added chemicals and polymers.

The endothermic dehydration of Ethanol to ethylene has been commercially practiced for many years. The early commercial reactor designs were based on isothermal, multi-tubular, shell-and-tube type arrangements in which the reactant ethanol would flow through the reactor tubes which would be individually packed with a fixed-bed catalyst. The required heat of reaction would be supplied by a circulating fluid such as molten salt, steam, Dowtherm, or some other fluid through the shell of the reactor. Another technology utilized expensive fired heaters handling ethanol to provide heat to reactants in multi stage reactor design. Because of the high temperatures required to effect the dehydration reaction and the high cost of fabrication of such reactors and fired heaters, the multi-tubular reactors were replaced by fixed-bed, adiabatic reactors which have been in commercial service since early 70's.

A number of patents on adiabatic reactors and process have been issued. British patent 516,360 which was granted in 1940 claims multiple reactor vessels connected in series. This patent further describes a heating arrangement to supply the required energy to each vessel for optimum temperature control. Furthermore, the patent provides provision for by-passing any vessel in order to control the production rate and/or perform necessary maintenance without having to shut down the process. Because of the adiabatic nature of the reaction, the temperature in any one stage decreases continuously as the dehydration reaction proceeds. To insure that the overall rate of reaction is maintained, inter-stage fired heating chambers are included between the successive reactor stages to provide the necessary thermal energy to sustain the reactions from stage to stage. This is a costly design. Additionally, the ethanol feedstock and the reaction products are continuously contacted with the hot surfaces within the fired heaters. The temperatures of the hot coils within the heaters are normally well above 400° C. which is needed to sustain the reactions within the reactor(s). This results in thermal decomposition of the valuable ethanol and products resulting in lower yield of ethylene and additionally the formation of impurities such as aldehydes, methane, and higher hydrocarbons etc. in the product.

U.S. Pat. No. 4,134,926 discloses a fluidized bed reactor concept for the dehydration of ethanol to ethylene wherein a portion of the dehydration catalyst is continuously withdrawn from the reactor chamber and regenerated with air in a second fluid-bed regenerator. The hot regenerated catalyst is then mixed with fresh make-up catalyst and recycled back to the primary reactor to provide the endodermic heat of reaction. This reactor concept has not found commercial application due to the complexity of the process, the handling and recycle of large quantities of solid catalyst, and continuous replacement of the lost catalyst because of attrition.

U.S. Pat. No. 4,232,179 describes a reactor train invention in which multiple, adiabatic reactor vessels are connected in series and/or parallel arrangement for dehydration of ethanol to ethylene. This patent further teaches the use of a sensible heat carrying fluid such as steam mixed with the ethanol feedstock prior to feeding to individual reactors. Each reactor is packed with a solid catalyst. The energy required for the reactions is supplied by a fired heater wherein both ethanol and steam are heated to very high temperatures needed for the reactions to proceed to completion in each reactor stage. This feature, being similar to British patent 516,360, can also result in lower selectivity and yield of the primary product and the formation of problematic by-products. In addition, no distinction is made in this disclosure as to the relative sizes of each reactor and the catalyst bed within that reactor with respect to other reactors and/or catalyst beds which make up the reactor train.

U.S. Pat. No. 4,396,789 teaches an invention which is basically similar to U.S. Pat. No. 4,232,179 with the exception that the reactor train is designed to operate at a design pressure of between 20 to 40 atmospheres. The patent claims that such high pressure operation will simplify the purification of the crude ethylene product during the subsequent cryogenic distillation to produce high quality ethylene for downstream applications.

In all the above processes, the dehydration catalyst was subjected to carbonization as a result of direct exposure of ethanol to high coil surface temperatures within the pre-heater. This practice would require frequent regeneration of the catalyst bed thus requiring downtime, loss of production, and shortened catalyst life.

It is the general object of this invention to maximize the utilization efficiency of the dehydration of ethanol feedstock to ethylene product while minimizing the production of undesirable by-products. The specific goal of the present invention is to provide a novel, adiabatic reactor configuration and process to achieve the desired goals of the invention. Other objects and benefits of the present invention will become apparent from the following disclosure. It is an object of the present invention to utilize available streams found within a facility or derived from the operation of the process carried out in the reactor. In this regard each of the stages, are independently sized and the quantity of catalyst therein determined to take advantage of a stream from some other reactor or source within the facility or from other stages within the reactor to obtain the highest yield and selectivity from these disparate sources. It is a particular object of the present invention to design each stage of the reactor considering various sources which can used in the reaction at hand.

### SUMMARY OF THE INVENTION

One aspect of the present invention is an adiabatic gas phase reactor comprising:

a) a plurality of stages, preferably having a substantially circular dimension, such as a cylinder

b) each of said stages having an independently determined internal diameter, length, and volume, and

d) each of said stages contains a quantity of fixed bed catalyst and inert support beds whose amount is independently determined, preferably wherein the independent determinations take into account the control of thermal energy the optimization of temperature profiles within the catalyst beds, and the feed rates of ethanol and inert gas to the individual stages of the reactor or external to the reactor to obtain the highest efficiency for ethanol conversion, ethylene selectivity, and yield. The stages can be arranged in series or parallel or in combinations thereof.

The stages, whether housed in a single structure or in separate structures, comprise a reactor train where the dehydration reaction or process is carried out. Each structure is designed to operate under different conditions of temperature, pressure, reactant residence time, and quantity of catalyst than the other structures.

### BRIEF DESCRIPTION OF THE DRAWINGS

FIG. 1 shows a single reactor vessel housing three catalyst zones in series with each zone having different length, diameter, volume, and/or catalyst quantity than the other zones and the flow arrangement of the reactant alcohol and the inert gas.

FIG. 2 depicts a second embodiment of the invention where three reactor vessels which are arranged in series comprise the reactor train with each vessel in this design has different length, diameter, volume, and/or catalyst quantity than the other vessels.

### DETAILED DESCRIPTION OF THE INVENTION

Recognizing (i) the short comings of the prior art as noted above, (ii) the key economic drivers needed for bio-ethylene production to compete as replacement for petroleum-derived ethylene, and (iii) the specific quality demands required of any bio-ethylene as feedstock for the traditional uses of these raw materials, this invention provides an improved reactor technology and process to specifically address these issues. This disclosure teaches a novel reactor design and geometry and an improved processing concept to achieve its desired goals. The novel reactor is configured according to two embodiments. In each embodiment, the multiple reactor vessels and/or reactor stages are employed in series or in parallel configuration wherein each stage and/or reactor vessel comprising the reactor train has a different internal diameter, length, volume, and quantity of fixed-bed catalyst than the other stages and/or vessels. Several improvements arise from this novel design whose detail is illustrated below. The number of stages is typically between 2 and 10 and preferably between 2 and 5. Each stage preferably has an internal diameter of between 0.5 to 10 meters at the inlet to the stage and an internal diameter of between 0.7 and 15 meters at the outlet of the stage with each stage preferably having a length of between 0.3 to 15 meters.

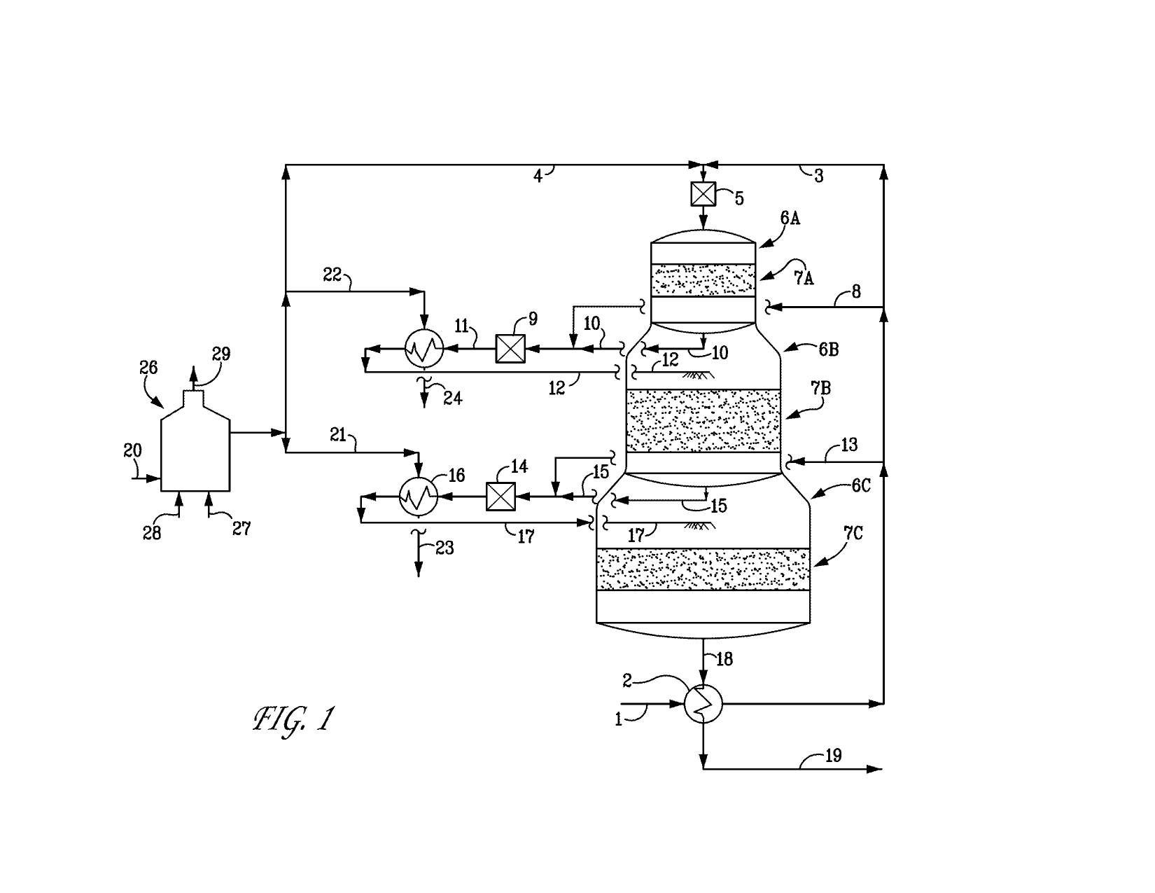

According to this disclosure, two embodiments of this process will be described in the following sections. FIG. 1 serves to illustrate one embodiment of this invention. In this particular embodiment, one reactor vessel containing three stages connected in series is employed. The three stages comprise the reactor vessel. Each stage is packed with a suitable fixed-bed dehydration catalyst such as those described in U.S. Pat. Nos. 4,260,845, 4,302,357, 4,529,827, 4,670,620, 4,873,392, and 6,489,515. Each stage in this arrangement has a different internal diameter, length, volume, and quantity of catalyst than the other stages. The variable sized stages are uniquely designed for a target production of ethylene. The benefits and the improvements made possible by this design will become obvious after the detailed explanation of the invention.

Hydrous or anhydrous ethanol stream 1 is vaporized and preheated in heat exchanger 2 using the hot reactor effluent gases 18 which then exit as stream 19 to downstream purification sections of the plant (not shown). The ethanol feed is not passed through a superheating furnace, but is separately pre-heated, to a temperature between 200° to 400° C. and mixed with the heat supplying inert gas in an in-line mixer prior to being introduced into any stage which is added to each stage at a rate of between 0.01 to 10 kg per hour per kg catalyst. and has a weight ratio of between 0.0 to 0.06 and preferably between 0.01 to 0.1 to the weight of the inert gas at the inlet to each stage. Within each stage as optimized according to the present invention, the operating temperature is from 300° C. to 550° C. and preferably from 350° C. to 500° C. at the inlet to each stage and wherein the outlet temperature of each stage is maintained at 250° C. to 500° C. and preferably from 300° C. to 450° C. at operating pressure of each stage is from 2 barg to 50 barg and preferably from 4 barg to 40 barg.

The hot stream 1 is then divided into three streams 3, 8, and 13. Stream 3 is combined with superheated inert gas stream 4 which is supplied by the inert super-heater 26 wherein cold inert gas enters via stream 20 and the heat source is provided by burning fuel supplied by stream 27 and air supplied by stream 28. Alternatively, the superheated inert gas can be supplied via other plant facilities such as a cogeneration plant. The combustion products from the super-heater 26 leave the stack via stream 29. The inline stationary mixer 5 serves to fully mix the pre-heated ethanol stream 3 and superheated stream 4 before entering the first stage reactor 6A. Stage 6A houses the fixed-bed bed 7A. The diameter, length, and the volume of catalyst in this stage is designed for optimum temperature profile and residence time of the ethanol reactant. Typically, the inlet temperature to this stage is between 400 to 550° C. and the outlet in the range of 300 to 480° C. The weight hourly space velocity (WHSV) of the ethanol in this stage is in the range of 0.01 to 10 kg ethanol per hour per kg of catalyst. Typically, the weight ratio of the inert gas to ethanol in the inlet to this stage is between 0.5 to 10 kg ethanol to inert gas. The operating pressure in this stage may range from 1(preferably 2) to 50 barg. These conditions are designed to optimize the temperature profile in this stage and to achieve complete conversion of ethanol and greater than 99% selectivity to the corresponding ethylene.

The exit stream 10 from stage 6A containing ethylene from stage 1 and water formed in stage 1 is mixed outside of the reactor vessel with fresh ethanol stream 8 in inline mixer 9 and heated to the desired temperature by exchanger 11. This exchanger is heated by superheated inert gas stream 22. The heat supplying inert gas to each reactor stage is superheated steam at pressure in the range of 1 to 50 barg and preferably 4 to 40 barg and at temperature in the range of 300° C. to 550° C. and preferably 350° to 500° C. The inert gas exit from heat exchanger 11 as stream 24 and is used for other heat requirement in the plant. Stream 12 from exchanger 11 is fed to second stage reactor 6B and is distributed downward to the catalyst bed 7B in this stage. The ranges of conditions in this second stage include: inlet temperature of 380-530° C., outlet temperature of 300-460° C., ethanol WHSV of 0.01 to 8 kg ethanol/hr/kg catalyst, ethanol-to-inert gas ratio of 0.8 to 15 kg ethanol/kg inert gas and pressure 2 to 50 barg. Again, the conditions are designed such that to obtain optimum temperature profile thought the catalyst bed, to achieve complete conversion of ethanol, and to realize >99% selectivity to ethylene.

The effluent stream 15 from second stage reactor is mixed with additional ethanol stream 13 in inline mixer 14, heated in exchanger 16. Exchanger 16 is heated by superheated inert gas 21. The heated stream 17 from exchanger 16 is the feed to reactor stage 6C which contains the 3^{rd} stage catalyst bed 7C. The operating conditions in this stage are also optimized to achieve similar goals of temperature profile and performance as in stages 6A and 6B. The ranges of conditions in this third stage include: inlet temperature of 370-520° C., outlet temperature of 290-420° C., ethanol WHSV of 0.01 to 6 kg ethanol/hr/kg catalyst, ethanol-to-inert gas ratio of 1 to 20 kg ethanol/kg inert gas, and pressure in the range 2 to 50 barg. The exit stream 18 from stage 6C flows to heat exchanger 18. Stream 19 containing crude ethylene, inert gas, and minor by-products exits this exchanger and is processed in downstream equipment for purification.

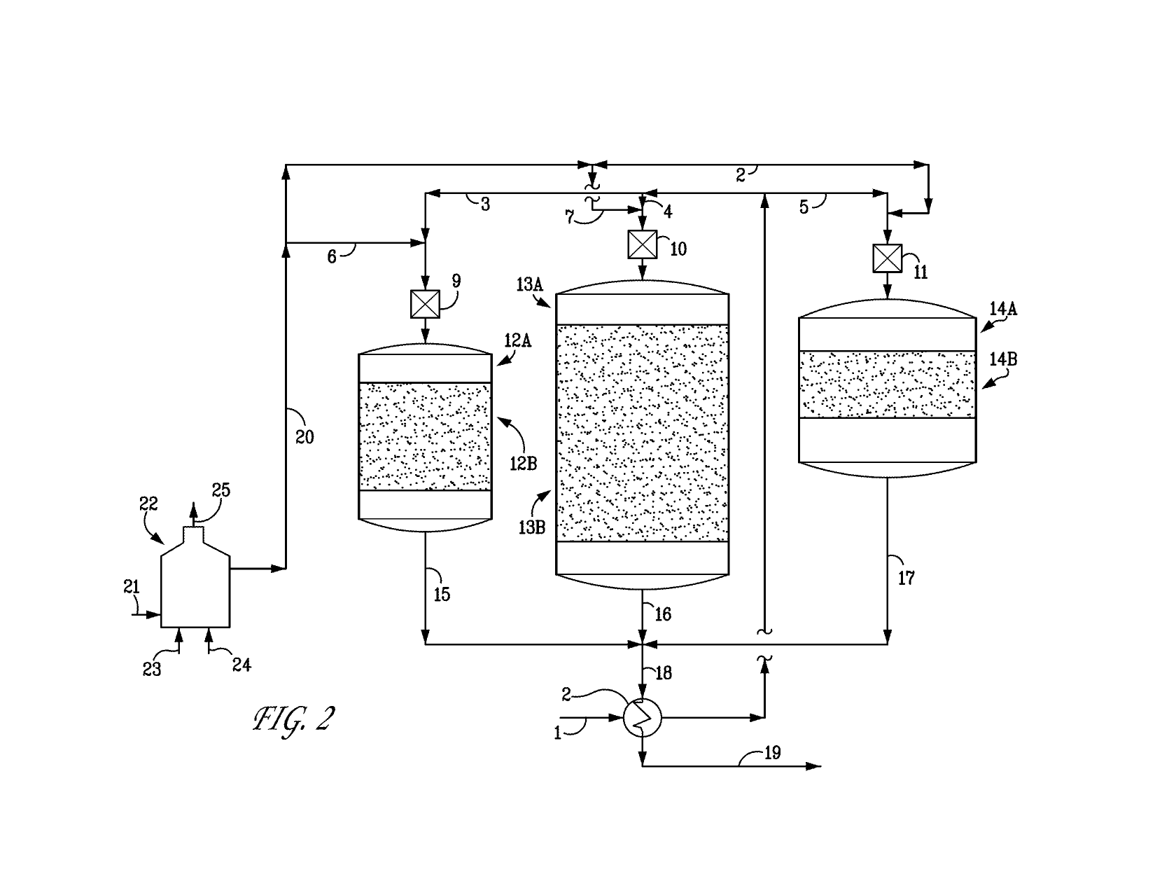

A second embodiment of the present invention is illustrated in FIG. 2. In this illustration, three reactor vessels are shown in parallel. The distinction between this variation and the variation shown in FIG. 1 is that, unlike the single reactor vessel with multiple catalyst stages and with each stage having different volume as shown in FIG. 1, there is one catalyst stage in each of the reactor vessels in the present variation, but effectively form a single reactor by purpose with the various stages interconnected to utilize the available streams. Similar to the previous variation, each vessel in this arrangement has a different internal diameter, length, volume than the other reactor vessels. In addition, the quantity of catalyst in each vessel is different than the quantity in other reactor vessels.

As FIG. 2 illustrates, the fresh ethanol stream 1 is vaporized and pre-heated in heat exchanger 2 prior to being divided into three streams 3, 4, and 5. Stream 3 is mixed with superheated inert gas stream 6 in inline mixer 9 and fed to the first reactor vessel 12A which contains the catalyst bed 12B. In similar fashion, stream 4 is mixed with superheated inert gas stream 7 in mixer 10 and fed to the second reactor vessel 13A which houses the catalyst bed 13B. Still in similar fashion, fresh ethanol stream ≡is mixed with superheated inert gas stream 8 in inline mixer 11. The mixture is fed to reactor vessel 14A which holds the catalyst bed 14B. The exits streams 15 from reactor vessel 12, exit stream 16 from reactor vessel 13A, and exit stream 17 from reactor vessel 14A are combined and heat exchanged in exchanger 18 before taken to ethylene recovery and purification sections of the plant (not shown).

The operating conditions within the individual reactor vessels in this arrangement are such that to achieve the desired performance criteria of optimum temperature profiles within the catalyst beds, complete conversion of ethanol feedstock, and >99% selectivity to ethylene product. The ranges for these conditions include the following. Typically, the inlet temperature to each reactor vessel is between 400 to 550° C. and the outlet in the range of 300 to 480° C. The weight hourly space velocity (WHSV) of the ethanol in each vessel is in the range of 0.01 to 10 kg ethanol per hour per kg of catalyst. The weight ratio of the inert gas to ethanol in the inlet to this stage is between 0.5 to 10 kg ethanol to inert gas. Finally, the operating pressure within each reactor vessel may range from 2 to 50 barg.

To those skilled in the art, the design features as detailed above offer major technical advances and make it possible to realize numerous improvements and advantages over the previous arts. These advances and improvements are noted in the following paragraphs.

As explained before, the catalytic dehydration of ethanol to ethylene is highly endothermic and requires considerable supply of energy to initiate the reaction and drive it to completion. The reaction produces one mole of water for each mole of ethanol reacted according to:

C₂H₅OH→C₂H₄+H₂O

This reaction requires about 400 kcal per kg of ethylene at the normal operating temperatures of 300° -400° C.

A competing reaction can also take place producing the undesirable by-product diethyl ether (DEE) according to the following reaction:

2C₂H₅OH→(C₂H₅)₂O+H₂O

The key is to minimize the formation of DEE and maximize the selectivity to ethylene product by the optimum arrangement and size of the reactor stages and the staged addition of ethanol and the heat supplying inert gas. Other by-products may also be formed by the secondary reaction of ethylene to other hydrocarbons such as dimerization to 1- and 2-butylene.

The kinetics of the primary reaction are very sensitive to the operating temperature regime within the catalyst bed. At the inlet to the reactor, the temperature has to be high enough to initiate the reactions. If the temperature gas mixture is too high at the inlet region, side reactions of ethanol will occur resulting in unwanted products. This reduces selectivity and yield to the desired ethylene product. As reactants pass through each catalyst bed, the temperature is continuously decreased toward the end of the catalyst bed. At the outlet of the catalyst bed, if the temperature is allowed to cool significantly because of inadequate supply of sensible energy, either ethanol conversion is not complete thus requiring recycle of ethanol or secondary reactions can occur resulting in unwanted by-products such as aldehydes. Therefore, the temperature profile through the catalyst bed is very critical to optimum performance.

Three design features in this invention combine to result in optimum temperature profile within the individual reactors. First, the multiple staging of the reactors into variable volume compartments allows for the optimum distribution and residence time of the reactant alcohol and inert gas through each stage. The variable volume is achieved by varying the internal diameter of each reactor stage, varying the length of each stage, and/or varying the volume of the catalyst bed within each stage. Stages may have continuously variable internal diameter from the inlet of the stage to the outlet of the stage. The optimization of volume and thus the residence time distribution of the reactants is an important consideration in the kinetics of the dehydration reaction and therefore the optimum utilization of the individual catalyst beds within the reactor stages.

Second, both the ethanol feed and the heat supplying inert gas to each stage are separately and independently fed, controlled, and heated prior to being mixed and distributed to the individual reactor stages. This makes it possible to avoid super-heating of ethanol and its thermal degradation. In addition, this feature allows the optimum utilization of the heat carrying inert gas in relation to the amount of ethanol feed rate. This optimization requires the balancing of sufficient energy supply to each stage but not excessive amounts which will result in economic disadvantage. The design also balances the formation of the water of reaction and the heat supplying inert gas. Furthermore, the design eliminates the formation of by-products such as DEE, aldehydes, or hydrocarbons such as propylene, butylenes, etc.

The third design feature stems from the resulting kinetics of the dehydration reaction made possible by realizing the complete conversion of ethanol through the individual reactor stages. Therefore, ethanol recovery and recycle are avoided in this processing scheme.

A further improvement of the present invention is that the economic life of each catalyst bed comprising the reactor train is considerably increased due to the optimum temperature profile within each stage. Therefore, frequent regenerations required in older technologies are avoided. The catalyst employed in this process may be alumina, silica-alumina, zeolites, or other suitable catalysts as are described in the patent literature. See for example Pat. Nos. 4,260,845, 4,302,357, 4,529,827, 4,670,620, 4,873,392, and 6,489,515. The longer catalyst life makes it possible for improved asset utilization and efficiency and allows for longer cycle time of the catalyst beds before unit shutdown and replacement are needed.

A further improvement resulting from the reactor design and the staged process for introducing ethanol feed and the heat supplying inert gas into each stage and/or reactor vessel is that each stage may be by-passed to control the production rate or make it possible to perform maintenance in that stage without losing efficiency or shutting down the whole process. Furthermore, this allows for partial or total removal of the catalyst bed in a particular stage without having to shut down the whole process.

In addition to the above improvements, other improvements can be readily realized from this invention by those experts familiar with the selective dehydration of ethanol to ethylene.

### EXAMPLES

The following experimental examples serve to illustrate the unique features of the present invention and the resulting performance of the dehydration system. An experimental pilot reactor was constructed to allow the simulation of the operating conditions within each reactor stage and the performance testing of the reactor design as taught in this invention. The reactor consisted of a 1 inch OD, 0.870 inch ID, 3.5 feet long fix-bed down flow reactor. The reactor was heated in a three-zone furnace whereby the temperature of each zone could be controlled independently to achieve a desired temperature profile within the catalyst bed. The reactor tube was equipped with a centrally positioned 3/16″ thermowell which housed five stationary thermocouples that were equally spaced within the thermowell at 0″, 2″, 4″, 6″, and 8″ measured from the top of the catalyst bed.

The catalyst used in these experiments was a commercially available high purity and surface area gamma alumina. Approximately 40 CC of this catalyst was loaded into the reactor. An equal volume of inert alpha alumina spheres was mixed with the active catalyst as diluent yielding a total bed volume of ˜80 CC. In addition, the same inert alumina spheres were used as pre- and post-heat zones of the reactor. The complete inertness of the spheres was demonstrated under all the operating conditions by testing the pilot reactor with only the alpha alumina packed inside the reactor tube.

The experimental setup was designed for continuous operation, sampling, and analysis of the products. The operating conditions were selected such that a two-stage design could be fully simulated and tested. The experimental conditions within the two stages were as shown in Table 1:

TABLE 1
Stage 1 and Stage 2 Experimental Conditions

| Operating Parameter | Stage 1 | Stage 2 |
|---|---|---|
| Pressure, barg | 6.45 | 5.95 |
| Inlet Temperature, ° C. | 466 | 453 |
| Outlet Temperature, ° C. | 375 | 374 |
| Feed Ethanol Conc., mole % | 8.75 | 7.60 |
| Feed Water Conc., mole % | 91.25 | 85.15 |
| Feed Ethylene Conc., mole % | 0 | 7.25 |
| Ethanol WHSV, g/hr/g cat. | 0.433 | 0.334 |

The performance measures in these tests included ethanol conversion, ethylene selectivity, and by-products analysis. The by-products included: methane, ethane, propylene, propane, methanol, acetaldehyde, 1-butane, 2-butane, acetone, diethyl ether, 1-pentene, 1-hexene, and n-hexane. The test results are summarized in Table 2.

TABLE 2
Stage 1 and Stage 2 Performance Data

| Performance Measure | Stage 1 | Stage 2 |
|---|---|---|
| Ethanol Conversion, % | 100 | 100 |
| Ethylene Selectivity, % | 100 | 100 |
| By-products Conc., % | ND | ND |

ND: Not Detected

The invention claimed is:

1. An adiabatic gas phase reactor vessel for application to catalytic dehydration of ethanol to ethylene process wherein:
a) the reactor vessel is comprised of several stages,
b) the stages are stacked in a series or parallel configuration,
c) each stage has a different internal diameter, length, and volume than the other stages,
d) each stage contains a quantity of fixed bed catalyst whose amount is different than the quantity in other stages within the reactor vessel, and
e) each stage is substantially circular.

2. The reactor vessel according to claim 1 wherein each stage has continuously variable internal diameter from the inlet of the stage to the outlet of the stage.

3. The reactor vessel according to claim 1 wherein:
the number of stages is between 2 and 10 and preferably between 2 and 5, each stage has an internal diameter of between 0.5 to 10 meters at the inlet to the stage and an internal diameter of between 0.7 and 15 meters at the outlet of the stage, and each stage has a length of between 0.3 to 15 meters.

4. An adiabatic gas phase process for catalytic dehydration of ethanol to ethylene process wherein:
a) the process is comprised of feeding ethanol through several dehydration stages,
b) the stages are stacked in a series or parallel configuration,
c) each stage has a different internal diameter, length, and volume than the other stages,
d) each stage contains a quantity of fixed bed catalyst whose amount is different than the quantity in other stages within the reactor vessel, and
e) each stage is substantially circular.

5. The process according to claim 4 wherein the ethanol feed:
is anhydrous or aqueous,
is not passed through a superheating furnace,
is separately pre-heated, to a temperature between 200° to 400° C. and mixed with the heat supplying inert gas in an in-line mixer prior to being introduced into any stage,
is added to each stage at a rate of between 0.01 to 10 kg per hour per kg catalyst, and
has a weight ratio of between 0.0 to 0.06 and preferably between 0.01 to 0.1 to the weight of the inert gas at the inlet to each stage.

6. The process according to claim 4 wherein:
the heat supplying inert gas to the each reactor stages is superheated steam at pressure in the range of 1 to 50 barg and at temperature in the range of 300° C. to 550° C.,
the operating temperature is from 300° C. to 550° C. at the inlet to each stage and the outlet temperature of each stage is maintained at 250° C. to 500° C., and
the operating pressure of each stage is from 2 barg to 50 barg.

7. The process according to claim 6 wherein the operating pressue in each stage is from 4 barg to 40 barg.

8. The process according to claim 4 wherein:
the heat supplying inert gas to each reactor stages is superheated steam at pressure in the range of 4 to 40 barg and at temperature in the range of 350° to 500° C., the operating temperature is from 350° C. to 500° C. at the inlet to each stage, the outlet temperature of each stage is maintained at from 300° to 450° C. and the operating pressure of each stage is from 4 barg to 40 barg.

9. An adiabatic reactor train configuration for application to the catalytic dehydration of ethanol to ethylene process wherein:
a) the train is comprised of several adiabatic gas phase reactor vessels,
b) the reactor vessels are connected in a series or parallel configuration,
c) each reactor vessel has a different internal diameter, length, and volume than the other vessels,
d) each reactor vessel contains a quantity of fixed bed catalyst whose amount is different than the quantity in other vessels, and
e) each vessel is substantially circular.

10. The reactor train according to claim 8 wherein each stage has continuously variable internal diameter from the inlet of the stage to the outlet of the stage.

11. The reactor train according to claim 9 wherein:
the number of reactor vessels is between 2 and 10 and preferably between 2 and 5, each reactor vessel has an internal diameter of between 0.5 to 10 meters at the inlet to the vessel and an internal diameter of between 0.7 and 15 meters at the outlet of the vessel, and each stage has a length of between 0.3 to 15 meters.

12. An adiabatic reactor train process for the catalytic dehydration of ethanol to ethylene process wherein:
a) the train process is comprised of feeding ethanol through several adiabatic gas phase dehydration reactor vessels,
b) the reactor vessels are connected in a series or parallel configuration,
c) each reactor vessel has a different internal diameter, length, and volume than the other vessels,
d) each reactor vessel contains a quantity of fixed bed catalyst whose amount is different than the quantity in other vessels, and
e) each vessel is substantially circular.

13. The process according to claim 12 wherein the ethanol feed:
is anhydrous or aqueous, p1 is not passed through a superheating furnace,
is separately pre-heated, to a temperature between 200° to 400° C. and mixed with the heat supplying inert gas in an in-line mixer prior to being introduced into any reactor vessel, is added to each vessel at a rate of between 0.01 to 10 kg per hour per kg catalyst, and
has a weight ratio of between 0.0 to 0.06 and preferably between 0.01 to 0.1 to the weight of the inert gas at the inlet to each vessel.

14. The process according to claim 12 wherein:
the heat supplying inert gas to each reactor vessel is superheated steam at pressure in the range of 1 to 50 barg at temperature in the range of 300° C. to 550° C., the operating temperature is from 300° C. to 550° C. at the inlet to each vessel and the outlet temperature of each vessel is maintained at 250° C. to 500° C., and the operating pressure of each vessel is from 2 barg to 50 barg

15. The process according to claim 12 wherein:
the heat supplying inert gas to each reactor vessel is superheated steam at pressure in the range of 4 to 40 barg and at temperature in the range of 350° to 500° C., the operating temperature is from 350° C. to 500° C. at the inlet to each vessel, the outlet temperature of each vessel is maintained at 300° C. to 450° C., and the operating pressure of each vessel is from 4 barg to 40 barg.

16. An adiabatic reactor train according to claim 9 wherein
at least one reactor vessel is comprised of several stages, the stages are stacked in a series or parallel configuration, each stage has a different internal diameter, length, and volume than the other stages, each stage contains a quantity of fixed bed catalyst whose amount is different than the quantity in other stages within the reactor vessel, and each stage is substantially circular.
